(19) United States
(12) Patent Application Publication
(10) Pub. No.: US 2009/0264771 A1
(43) Pub. Date: Oct. 22, 2009

(54) Ultrasonic Based Characterization of Plaque in Chronic Total Occlusions

(75) Inventors: Richard HOUBEN, Lanaken (BE); Sean WHELAN, Knockdog (IE); Niall DUFFY, Tuam (IE); Ashish VARMA, Kinvara (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/107,536

(22) Filed: Apr. 22, 2008

(51) Int. Cl. A61B 8/00

(52) U.S. Cl. 600/470

(57) ABSTRACT

A probe for detecting at least one property of a chronic total occlusion in a lumen. The probe includes an elongated member having a distal end configured to be inserted into the lumen, and an ultrasound transducer mounted at the distal end of the elongated member. The ultrasound transducer is configured to emit an ultrasonic signal and to receive a reflected signal from the chronic total occlusion. The probe also includes a processor configured to receive the reflected signal from the ultrasound transducer and convert the reflected signal to at least one property of the chronic total occlusion.

### BACKGROUND OF THE INVENTION

1. Field of the Invention

The present invention is generally related to a probe for characterizing properties of plaque in a chronic total occlusion.

2. Background of the Invention

Stenotic lesions may comprise a hard, calcified substance and/or a softer thrombus material, each of which forms on the lumen walls of a blood vessel and restricts blood flow there through. Intra-luminal treatments such as balloon angioplasty (PTA, PTCA, etc.), stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees throughout its length. In order to place the distal end of a catheter at the treatment site, a guidewire is typically introduced and tracked from an incision, through the vasculature, and across the lesion. Then, a catheter (e.g. a balloon catheter), perhaps containing a stent at its distal end, can be tracked over the guidewire to the treatment site. Ordinarily, the distal end of the guidewire is quite flexible so that it can be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion.

Thus, for treatment of total occlusions, stiffer guidewires have been employed to recanalize through the total occlusion. However, due to the fibrous cap of the total occlusion, a stiffer guidewire still may not be able to cross the occlusion. When using a stiffer guidewire, great care must be taken to avoid perforation of the vessel wall.

Further, in a CTO, there may be a distortion of the regular vascular architecture such that there may be multiple small non-functional channels throughout the occlusion rather than one central lumen for recanalization. Thus, the conventional approach of looking for the single channel in the center of the occlusion may account for many of the failures. These spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Another equally important failure mode, even after a guidewire successfully crosses a chronic total occlusion, is the inability to advance a balloon or other angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion.

Atherosclerotic plaques vary considerably in their composition from site to site, but certain features are common to all of them. They contain many cells; mostly these are derived from cells of the wall that have divided wildly and have grown into the surface layer of the blood vessel, creating a mass lesion. Plaques also contain cholesterol and cholesterol esters, commonly referred to as fat. This lies freely in the space between the cells and in the cells themselves. A large amount of collagen is present in the plaques, particularly advanced plaques of the type which cause clinical problems. Additionally, human plaques contain calcium to varying degrees, hemorrhagic material including clot and grumous material composed of dead cells, fat and other debris. Relatively large amounts of water are also present, as is typical of all tissue.

Successful recanalization of chronic total occlusions remains an area where improvements are needed. Approximately 30% of all coronary angiograms in patients with coronary artery disease will show a CTO and its presence often excludes patients from treatment by percutaneous coronary intervention. Acute success rates vary according to the duration of occlusion, the morphology of the lesion and the coronary anatomy, the experience of the operator, the degree of persistence employed, and the type of equipment used. Recanalization rates range between 45-80%, with the highest success in short, recently occluded (<1 month), non-calcified lesions.

It is desirable to be able to characterize the plaque in the CTO before attempting to cross the CTO with a guidewire to minimize potential trauma to the area of the lumen at or near the CTO. By being able to characterize the plaque, softer regions may be identified, which may increase the chance of success of crossing the CTO with a guidewire without damaging surrounding tissue.

### SUMMARY OF THE INVENTION

The present invention describes an apparatus and method to characterize at least one property of a CTO and to visualize and track soft spots/channels through a CTO by characterizing at least one property of the CTO. The apparatus and method use ultrasound for invasive real time characterization of plaque in chronic total occluded coronary arteries.

According to an aspect of the present invention, there is provided a probe for detecting at least one property of a chronic total occlusion in a lumen. The probe includes an elongated member having a distal end configured to be inserted into the lumen, and an ultrasound transducer mounted at the distal end of the elongated member. The ultrasound transducer is configured to emit an ultrasonic signal and to receive a reflected signal from the chronic total occlusion. The probe also includes a processor configured to receive the reflected signal from the ultrasound transducer and convert the reflected signal to at least one property of the chronic total occlusion.

According to an aspect of the invention, there is provided a method for detecting at least one property of a chronic total occlusion in a vessel. The method includes steering an elongated member having an ultrasound transducer at a distal end thereof to a location in the vessel proximal to the chronic total occlusion, emitting an ultrasonic signal towards a portion of the chronic total occlusion, receiving a signal reflected from the portion of the chronic total occlusion, and correlating the received signal to at least one property of the chronic total occlusion.

According to an aspect of the invention, there is provided a method for traversing a chronic total occlusion. The method includes a) steering an elongated member having an ultrasound transducer at a distal end thereof to a location in the vessel proximal to the chronic total occlusion; b) emitting an ultrasonic signal from the ultrasound transducer towards a portion of the chronic total occlusion; c) receiving a signal reflected from the portion of the chronic total occlusion; d) correlating the received signal to a hardness of the chronic total occlusion; e) repositioning the elongated member to another location in the vessel; f) determining a soft spot in the chronic total occlusion based on repeating a)-e); g) penetrating a guidewire into the soft spot of the chronic total occlusion; and h) detecting penetration of the guidewire with bioelectrical impedance.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 is a schematic diagram of a vessel with a CTO;

FIG. 2 is a schematic diagram of a probe according to embodiments of the present invention;

FIG. 3 is a enlarged perspective view of an embodiment of a distal end of an elongated member of the probe of FIG. 2 having an ultrasound transducer mounted thereon;

FIG. 4 is a schematic diagram of the elongated member of FIG. 3 located within the vessel of FIG. 1 at different positions;

FIGS. 5A and 5B are graphs representing output signals of the ultrasound transducer as a function of time;

FIG. 6 is a schematic embodiment of a hardness map of the CTO of FIG. 1 generated by the probe of FIG. 2;

FIG. 7 is an enlarged perspective view of an embodiment of the distal end of the elongated member having a plurality of ultrasound transducers mounted thereon;

FIG. 8 is an enlarged perspective view of an embodiment of the distal end of the elongated member having an annular array of ultrasound transducers mounted thereon;

FIG. 9 is a schematic view of the probe that is configured to detect an electrocardiogram of a patient;

FIG. 10 is a schematic view of an embodiment of the probe that also measures bioelectrical impedance;

FIG. 11 is an enlarged perspective views of an embodiment of a distal end of a guide catheter having a plurality of ultrasound transducers mounted thereon; and

FIG. 12 is an enlarged perspective view of an embodiment of a distal end of a guide catheter having a plurality of ultrasound transducers mounted thereon.

### DETAILED DESCRIPTION OF EMBODIMENTS OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

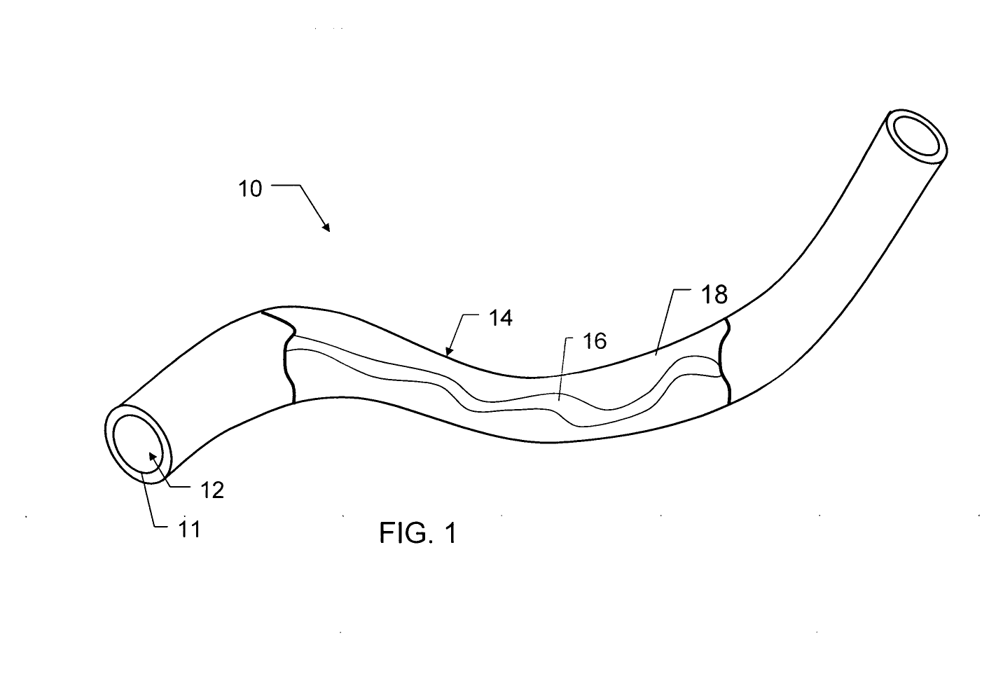

FIG. 1 illustrates a vessel 10 having an inner wall 11 that defines a lumen 12, and a chronic total occlusion (CTO) 14 within the lumen 12. The CTO 14 substantially or completely blocks flow of blood through the vessel 10. The CTO 14 includes a soft region of plaque 16 and a hard region of plaque 18 that substantially surrounds the soft region of plaque 16. The terms “soft” and “hard” are relative terms that are used to generally characterize the plaque in the CTO and are not defined by absolute values of hardness. A soft region of plaque is generally considered to be a region through which a guidewire may pass with little resistance. A hard region of plaque is generally considered to be a region that a guidewire cannot penetrate by normal methods, i.e. by simply pushing the guidewire against the hard region. Identifying where the soft region 16 of the CTO 14 is specifically located may significantly assist a clinician in determining where to attempt to push the guidewire through the CTO 14.

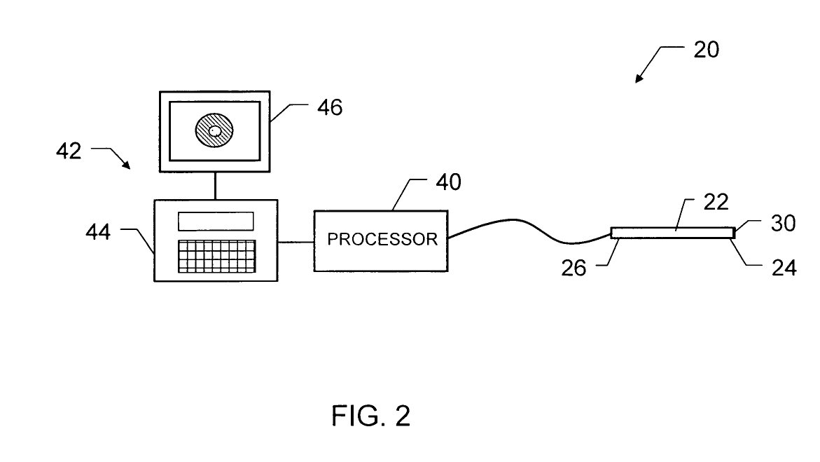

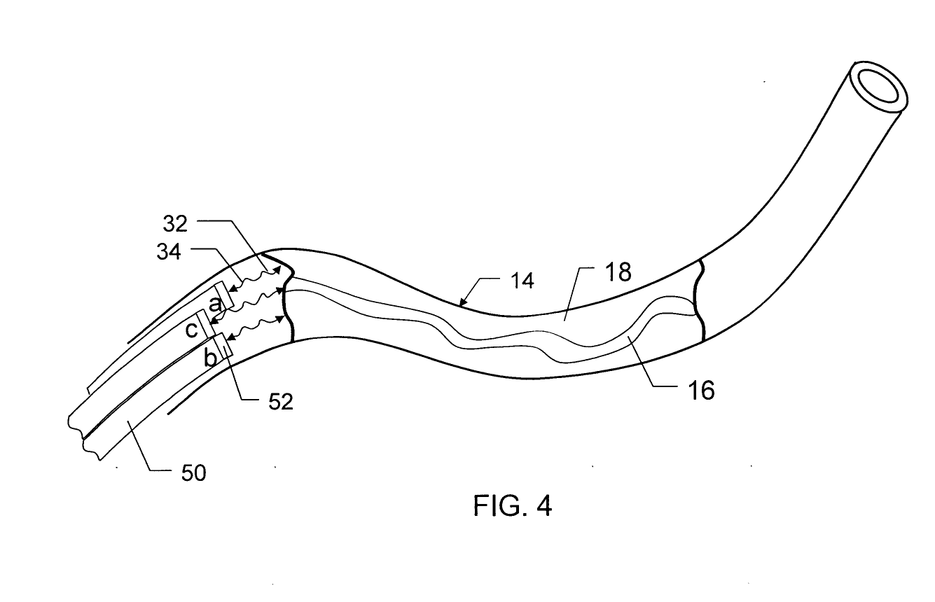

FIG. 2 illustrates an embodiment of a probe 20 for detecting at least one property of the CTO 14 in the lumen 12 of FIG. 1. As illustrated, the probe 20 includes an elongated member 22 that is configured to enter and advance in the lumen 12 to the CTO 14. The elongated member 22 has a distal end 24 that enters the lumen 12, and a proximal end 26 that remains outside of the lumen 12. The probe 20 also includes an ultrasound transducer 30 that is mounted at the distal end 24 of the elongated member 22. As discussed in further detail below, the ultrasound transducer 30 is configured to emit an ultrasonic signal 32 towards the CTO 14 and to receive a reflected signal 34 from the CTO 14, as shown in FIG. 4.

The probe 20 also includes a processor 40 that is connected to the proximal end 26 of the elongated member 22 and a user interface 42 that is connected to the processor 40. The processor 40 is configured to provide a signal to the ultrasound transducer 30 so that the transducer 30 will emit the ultrasonic signal 32. The processor 40 is also configured to receive the reflected signal 34 from the transducer 30 and convert the reflected signal to at least one property of the CTO 14, as discussed in further detail below.

The user interface 42 may include one or more input devices 44 that allow the operator of the probe 20 to turn the probe 20 on and off, adjust settings, trigger the processor 30 to provide the signal to the transducer 30, etc. The input devices 44 may include buttons, switches, knobs, or any other suitable devices that allow the operator to change a condition of the probe 20. The user interface 42 also includes one or more output devices 46 that are configured to provide the operator with information about the probe 20 and the CTO 14. For example, the output devices 46 may include a video monitor that provides graphics representing at least one property of the CTO 14 being measured by the probe 20, and lights representing a condition of the probe 20, e.g., whether the probe is on or off, etc.

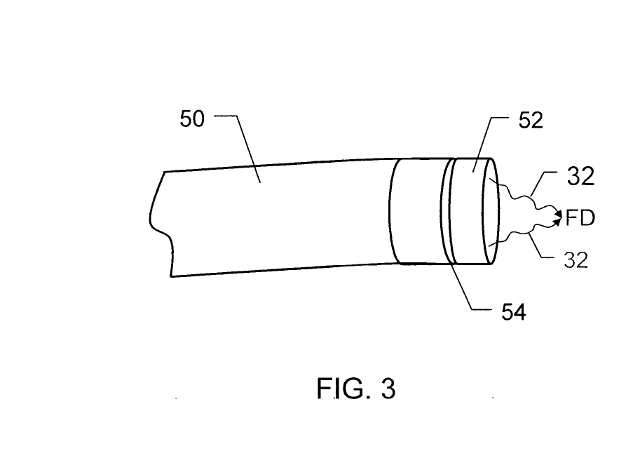

FIG. 3 illustrates an embodiment of the probe 20 in which the elongated member 22 is a guidewire 50 and the ultrasound transducer 30 is a single element ultrasound transducer 52 that is mounted on the guidewire 50 at a distal end 54 thereof. The transducer 52 may have a concave shape that is configured to allow the ultrasonic signal 32 that is provided by the transducer 52 to be focused at a predefined focal depth FD. The transducer 52 may include a piezoelectric crystal that is configured to oscillate at a high frequency when voltage is provided by the processor 40, thereby emitting the ultrasonic signal 32. In an embodiment, the piezoelectric crystal comprises lead zirconate titanate, although any suitable material may be used. In an embodiment, the transducer 52 may include a plurality of piezoelectric micromachined ultrasound transducers.

In operation, the probe 20 provides the clinician with continuous information on at least one property, such as the hardness or composition, of the CTO 14 that is just distal to the distal end 54 of the guidewire 50. FIG. 4 illustrates the guidewire 50 with the mounted ultrasound transducer 52 being manually positioned at three locations, represented by a, b, c. At each location a, b, c, the ultrasonic signal 32 is provided to the CTO 14 by the transducer 52 and the reflected signal 34 is received be the transducer 52 and communicated to the processor 40 for signal processing. The processor 40 may be programmed to output the reflected signal 34 over time to the output device 46 of the user interface 42 so that the reflected signal 34 may be displayed as a function of time.

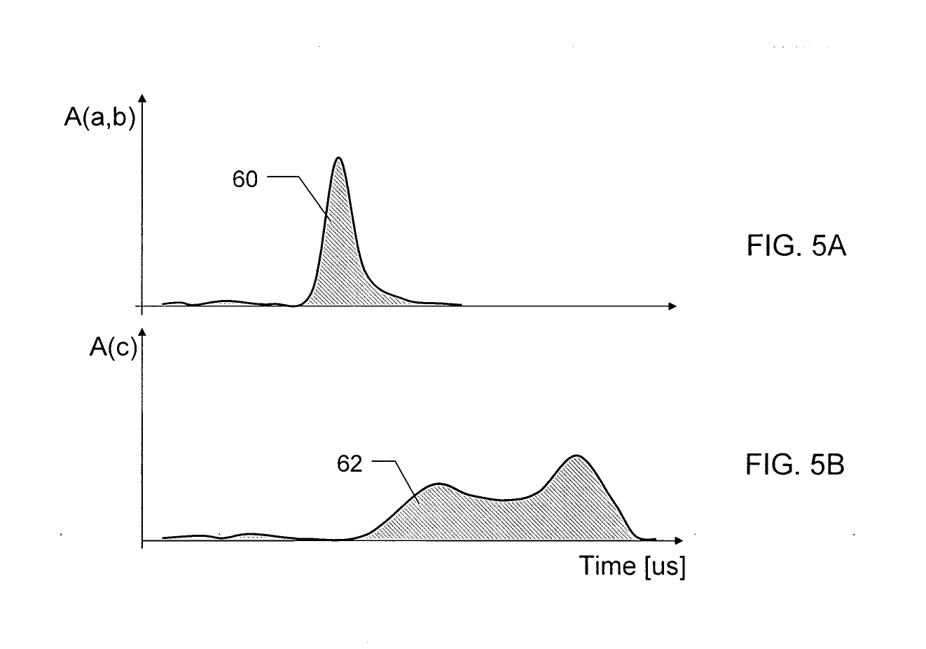

FIGS. 5A and 5B illustrate the difference in the reflected signals 34 that are measured by the transducer 52 at the three locations a, b, c shown in FIG. 4. FIG. 5A illustrates a reflected signal 60 at locations a and b, and FIG. 5B illustrates a reflected signal at location c. Both Figures show the amplitudes, represented by A, of the ultrasound echoes, i.e., reflected signals 34, as a function of time. The reflected signal 60 of FIG. 5A has relatively high amplitude ultrasound echoes within a narrow time frame, as compared to the reflected signal 62 of FIG. 5B, which has relatively low amplitude ultrasound echoes spread out in time. The reflected signal 60 indicates that there is relatively hard plaque 18 at locations a, b, and the reflected signal 62 indicates that there is relatively soft plaque 16 at location c.

Analysis of the reflected signals 60, 62 may also provide information on the composition of the plaque. Identification of an area of the CTO 14 that includes soft plaque 16, i.e., a “soft spot”, provides information to the clinician on the location to enter the CTO 14. Once the transducer 52 has entered the CTO, further information on the composition of the CTO 14 may be provided by continuing CTO 14 characterization, i.e., measuring the reflected signal 34 received by the transducer 52 over time. For example, if the probe 20 indicates a transition from soft plaque 16 to hard plaque 18, the transducer 52 may be at a dead-end or at a curve of the lumen 12 and CTO 14, which would indicate that the guidewire 50 would need to be steered in a different direction to continue to traverse the CTO.

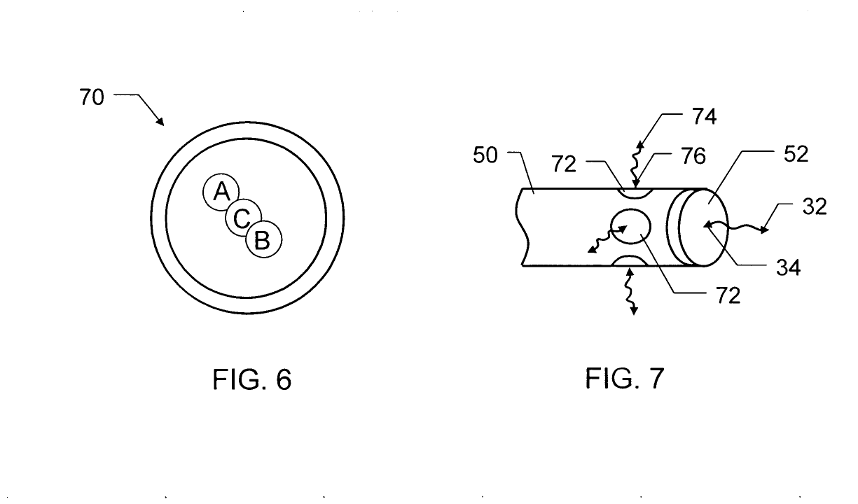

In an embodiment, the transducer 52 may be used to create a hardness “map” of plaque formation, such as the CTO 14, within the lumen 12. An example of a hardness map 70 is shown in FIG. 6, with A, B, and C representing the levels of hardness measured with the guidewire 50 being positioned at locations a, b, c, respectively, in FIG. 4. Due to cardiac wall movement of the vessel, such as when the vessel is a coronary artery, the transducer 52 may be randomly positioned within the lumen 12 of the vessel 10 at different locations, such as locations a, b, c in FIG. 4. The guidewire 50 may also be intentionally positioned in random locations by the clinician. By providing continuous measurement of the position of the distal end 54 of the guidewire 50 within the lumen 12 and, at the same time, the hardness of plaque that has formed within the lumen 12 of the vessel 10, the hardness map 70 may be created by the processor 40 and output to the user interface 42 for display.

The position of the distal end 54 of the guidewire 50 within the lumen 12 may be measured by at least two additional ultrasound transducers 72, shown in FIG. 7, that are positioned radially orthogonal to each other and orthogonal to the transducer 52. The transducers 72 are configured to measure the distance between the transducers 72 and the inner wall 11 of the vessel 10 that defines the lumen 12 by emitting ultrasonic signals 74 to the inner wall 11 and receiving reflected signals 76 from the inner wall 11. The processor 40 may be used to correlate the reflected signals 76 to a distance by known methods. The processor 40 may also be programmed to create the hardness map 70, based on the reflected signals 34, 76 and output the hardness map 70 to the output device 46 of the user interface 42.

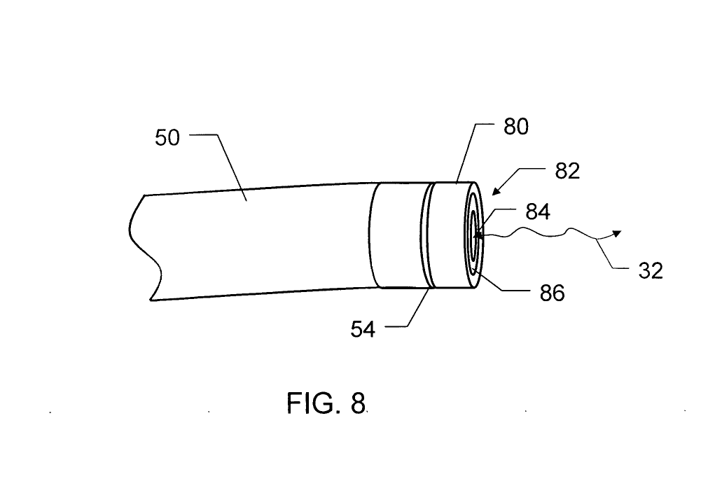

FIG. 8 illustrates an embodiment of an ultrasound transducer 80 located at the distal end 54 of the guidewire 50 that includes an annular array 82. The annular array 82 includes a center element 84 and at least one concentric element 86. The annular array 82 may permit active focusing of the emitted ultrasonic signals 32 within a range of depths, as compared to the single element transducer 52 that provides focal operation at only one depth. In an embodiment, three or four concentric elements and a center electrode may be included in the annular array 82, particularly for applications where a transducer and guidewire having larger diameters may be used. The annular array may include piezoelectric crystals, such as lead zirconate titanate or any other suitable material. In an embodiment, the annular array includes piezoelectric micromachined ultrasound transducers.

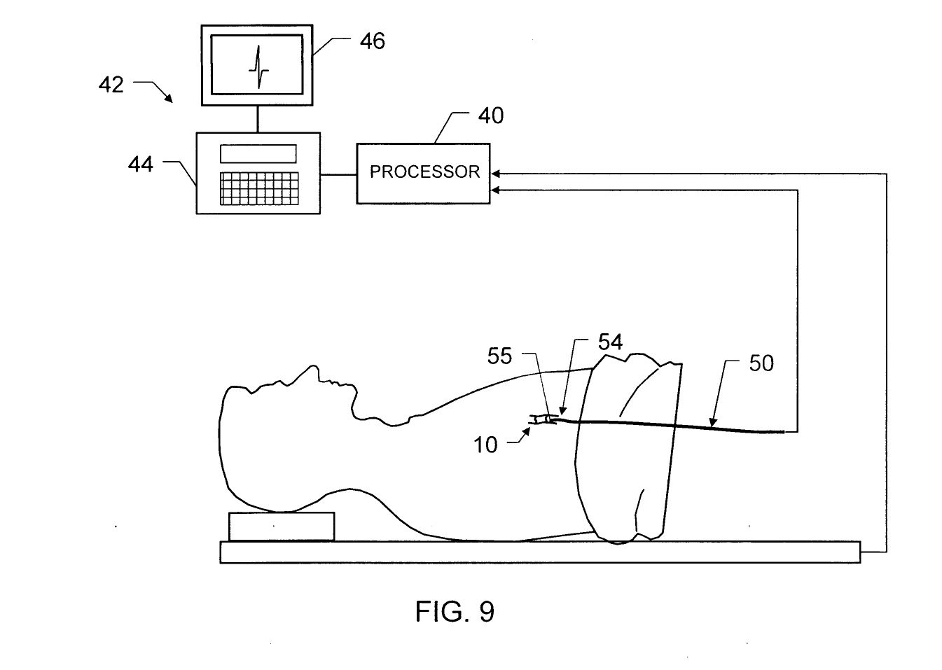

After the guidewire 50 enters the CTO 14 through a soft spot, i.e., a relatively soft area of plaque as detected by the probe 20, contact with the wall 11 of the vessel may be detected by continuously recording an electrocardiogram (“ECG”). As illustrated in FIG. 9, the ECG may be detected by using a sensor 55 located at the distal end 54 of the guidewire 50, which is conductive, with respect to the patient table as an indifferent reference, which provides a unipolar recording mode. The ECG may be output by the output device 46 of the user interface 42. A sudden ECG amplitude and/or morphology change may be detected and interpreted as wall contact by the distal end 54 of the guidewire 50. Wall contact may provide feedback to the clinician to pull back the guidewire 50, due to contact with the wall 11 of the vessel 10 or entry into what is commonly known as a “false lumen” or “phantom entrance,” and find another area of soft plaque 16 in the CTO 14 so that the wall 11 is not penetrated by the guidewire 50. The sensor 55 may also be used with the ECG to detect the wall 11 of the vessel 10 as the distal end 54 of the guidewire 50 is steered within the lumen 12 to the CTO 14.

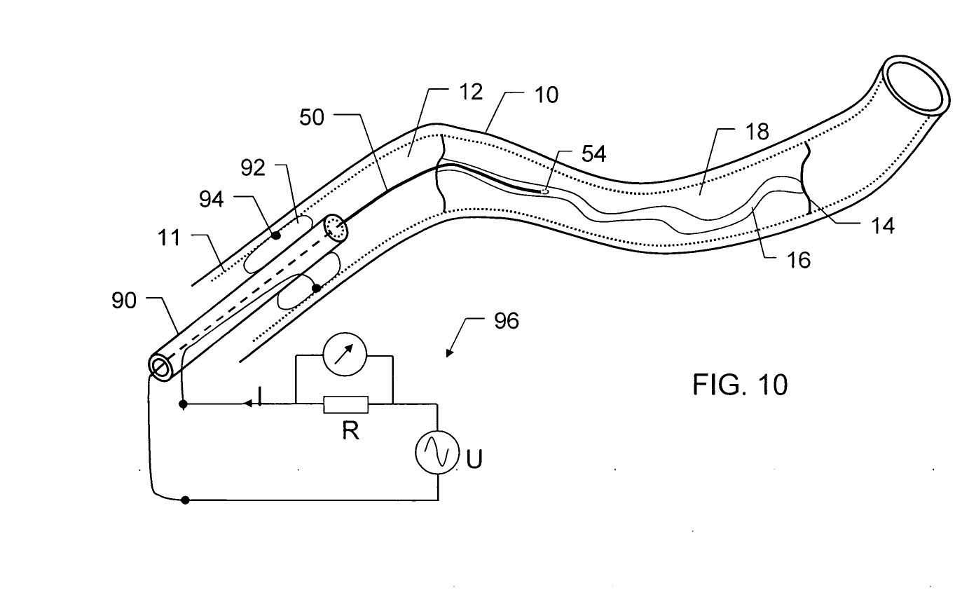

FIG. 10 illustrates an embodiment of the probe 20 in which a bioelectrical impedance measurement is used prevent the guidewire 50 from passing through the wall 11 of the vessel 10. The guidewire 50 may provide feedback to the clinician that indicates that the wall 11 of the vessel 10 has been reached by the distal end 54 of the guidewire 50. In response to the indication, the clinician may pull back on the guidewire 50 so that the wall 11 of the vessel 10 is not penetrated. The guidewire 50 may then be steered in a different direction.

The impedance may be measured between the distal end 54 of the guidewire 50, which is insulated, and at least one conductive element 94. An example of a system that uses bioelectrical impedance to guide a flexible elongated transluminal device through an occlusion is described in United States Patent Application Publication No. 2007/0255270, which is incorporated by reference in its entirety.

As illustrated in FIG. 10, a catheter 90 having a balloon 92 mounted thereon may be tracked into the lumen 12 over the guidewire 50. The balloon 92 may include at least one conductive element 94, which may be in the form of a conductive ring in this embodiment. A cross-section of the conductive ring on the inflated balloon 92 is shown in FIG. 9.

The impedance may be measured between the distal end 54 of the guidewire 50 and the conductive element 94 on the balloon 92, once the balloon 92 has been inflated against the inner wall 11 of the lumen 12 of the vessel 10. An alternating current I may be used to prevent hydrolysis and stimulation of the heart, and may be provided by a circuit 96 shown in FIG. 10. As illustrated, the circuit provides the alternating current I with an alternative voltage source U and a resistor R. Other circuits may be used and the circuit illustrated is provided only as an example and should not be considered to be limiting in any way.

In an embodiment, the at least one conductive element 94 may be a bipolar pair of electrodes (not shown) that may be provided at the distal end 54 of the guidewire 50. The electrodes may be ring electrodes and spaced apart at a distance so that the inner ring electrode is located at the distal end 54 of the guidewire 50, and the outer ring electrode is configured to contact the wall 11 of the vessel 10. In such an embodiment, the balloon 92 of FIG. 10 is not needed, because impedance is measured between the distal end 54 of the guidewire 50 and the outer electrode.

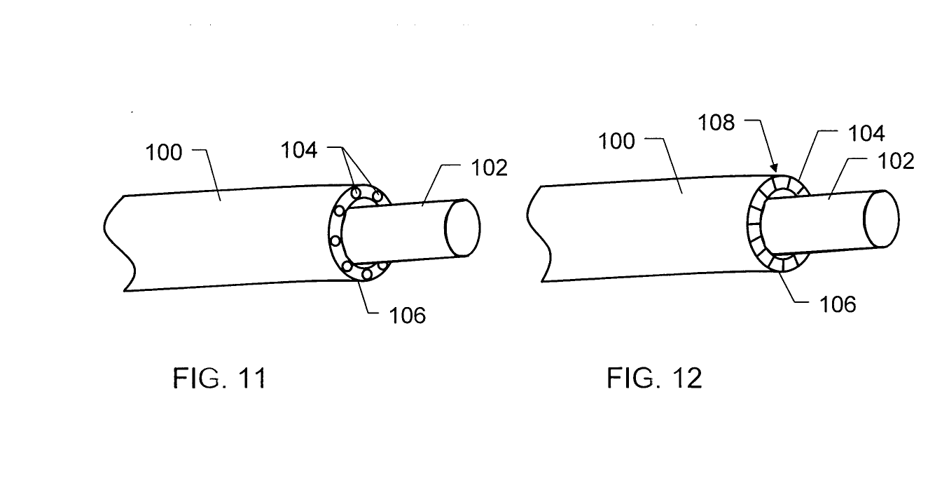

FIGS. 11 and 12 illustrate embodiments of the probe 20 in which the elongated member 22 is a guide catheter 100 that is configured to track over a guidewire 102. In this embodiment, the ultrasound transducer 30 is mounted on the guide catheter 100 instead of the guidewire 102. As illustrated, a plurality of ultrasound transducer elements 104 may be mounted on a distal end 106 of the guide catheter 100. The ultrasound transducer elements 104 may be used to characterize at least one property, such as hardness and/or composition, of the CTO 14. The ultrasound transducer elements 104 may be separate elements, as shown in FIG. 11, or may be in a circular array 108 of diced elements on a common base of piezoelectric or composite material, as shown in FIG. 12.

The piezoelectric material may include lead zirconate titanate, but could also be any other piezoelectric material, including capacitive micromachined ultrasound transducers. In an embodiment, as many as four micromachined ultrasound transducers may be provided in the circular array 108 to allow the ultrasonic signals to be steered. In this way, the CTO 14 can be characterized by multiple elements that create a circumferential view of the CTO 14 so that the locations of the soft plaque 16 and the hard plaque 18 may be identified and output to the user interface 50.

In an embodiment, the guidewire 50 discussed above may be used with the guide catheter 100. Such an arrangement would allow the position of the guidewire 50 that is equipped with the single element ultrasound transducer 52, or the ultrasound transducer 80 having the annular array of transducers 82, to be monitored by the ultrasound transducer elements 104 on the guide catheter 100. By combining this with the CTO hardness map 70 obtained by ultrasound characterization, information may be provided to the clinician on where the distal end of the guidewire is currently located and where the distal end of the guidewire should be. In other words, the clinician may be given guidance on how to reposition the guidewire towards a CTO “soft spot”. Imaging the lumen 12 of the vessel 10 may provide additional information on the localization of the catheter.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

What is claimed is:

1. A probe for detecting at least one property of a chronic total occlusion in a lumen of a patient, the probe comprising:
an elongated member having a distal end configured to be inserted into the lumen;
an ultrasound transducer mounted at the distal end of the elongated member, the ultrasound transducer being configured to emit an ultrasonic signal and to receive a reflected signal from the chronic total occlusion; and
a processor configured to receive the reflected signal from the ultrasound transducer and convert the reflected signal to at least one property of the chronic total occlusion.

2. A probe according to claim 1, wherein the at least one property comprises a hardness of the chronic total occlusion.

3. A probe according to claim 1, wherein the ultrasound transducer comprises a concave surface configured to focus the ultrasound signal at a predefined focal length.

4. A probe according to claim 1, wherein the ultrasound transducer comprises a piezoelectric material.

5. A probe according to claim 4, wherein the piezoelectric material comprises lead zirconate titanate.

6. A probe according to claim 1, wherein the ultrasound transducer comprises a plurality of micromachined ultrasound transducers.

7. A probe according to claim 1, further comprising a second ultrasound transducer mounted to the elongated member near the distal end, the second ultrasound transducer being configured to emit a second ultrasonic signal and to receive a second reflected signal from an inner wall of the lumen.

8. A probe according to claim 7, wherein the second ultrasound transducer is positioned orthogonal to the ultrasound transducer.

9. A probe according to claim 7, further comprising a third ultrasound transducer mounted to the elongated member near the distal end, the third ultrasound transducer being configured to emit a third ultrasonic signal and to receive a third reflected signal from the inner wall of the lumen.

10. A probe according to claim 7, wherein the processor is configured to receive the second reflected signal from the second ultrasound transducer and to create a hardness map of the chronic total occlusion based on the reflected signal and the second reflected signal, representative of hardness of the chronic total occlusion and position of the measured hardness, respectively.

11. A probe according to claim 1, wherein the ultrasound transducer comprises an annular array of piezoelectric materials, the array being configured to focus the ultrasonic signal within a range of depths.

12. A probe according to claim 1, wherein the elongated member is a guidewire.

13. A probe according to claim 1, wherein the elongated member is a catheter.

14. A probe according to claim 1, further comprising a sensor at the distal end of the elongated member, the sensor being configured to detect an electrocardiogram of the patient and provide feedback when the distal end of the elongated member contacts a wall that defines the lumen.

15. A probe according to claim 1, further comprising an impedance measurement circuit constructed and arranged to provide feedback when the distal end of the elongated member contacts a wall that defines the lumen.

16. A probe according to claim 15, wherein the impedance measurement circuit comprises a conductive element constructed and arranged to contact the wall that defines the lumen.

17. A probe according to claim 16, further comprising a catheter having a balloon mounted thereon, wherein the balloon comprises the conductive element.

18. A method for detecting at least one property of a chronic total occlusion in a vessel of a patient, the method comprising:
steering an elongated member having an ultrasound transducer at a distal end thereof to a location in the vessel proximal to the chronic total occlusion;
emitting an ultrasonic signal towards a portion of the chronic total occlusion;
receiving a signal reflected from the portion of the chronic total occlusion; and
correlating the received signal to at least one property of the chronic total occlusion.

19. A method according to claim 18, wherein the at least one property comprises a hardness of the portion of the chronic total occlusion.

20. A method according to claim 18, further comprising:
repositioning the elongated member to a second location in the vessel;
emitting another ultrasonic signal towards a second portion of the chronic total occlusion;
receiving a second signal reflected from the second portion of the chronic total occlusion;
correlating the received second signal to at least one property of the chronic total occlusion; and
generating a property map of the chronic total occlusion as a function of the location of the elongated member in the vessel.

21. A method according to claim 20, wherein the at least one property of the chronic total occlusion comprises a hardness of the chronic total occlusion, and wherein the property map is a hardness map.

22. A method according to claim 18, further comprising detecting a wall of the vessel using bioelectrical impedance during said steering.

23. A method according to claim 18, further comprising detecting a wall of the vessel using an electrocardiogram of the patient during said steering.

24. A method for traversing a chronic total occlusion, the method comprising: a) steering an elongated member having an ultrasound transducer at a distal end thereof to a location in the vessel proximal to the chronic total occlusion;
b) emitting an ultrasonic signal from the ultrasound transducer towards a portion of the chronic total occlusion;
c) receiving a signal reflected from the portion of the chronic total occlusion;
d) correlating the received signal to a hardness of the chronic total occlusion;
e) repositioning the elongated member to another location in the vessel;
f) determining a soft spot in the chronic total occlusion based on repeating
a)-e); and
g) penetrating a guidewire into the soft spot of the chronic total occlusion.

25. A method according to claim 24, further comprising detecting a wall of the vessel using bioelectrical impedance.

26. A method according to claim 24, further comprising detecting a wall of the vessel using an electrocardiogram of the patient.
